(19) United States
(12) Patent Application Publication
(10) Pub. No.: US 2012/0060833 A1
(43) Pub. Date: Mar. 15, 2012

(54) INHALATION THERAPY DEVICE

(75) Inventor: Wolfgang Achtzehner, Alling (DE)

(73) Assignee: PARI Pharma GmbH, Stamberg (DE)

(21) Appl. No.: 13/202,485

(22) PCT Filed: Feb. 19, 2010
(86) PCT No.: PCT/EP2010/052128

§ 371 Date: Nov. 7, 2011

(30) Foreign Application Priority Data
Feb. 20, 2009 (DE) 10 2009 001 037.8

(51) Int. Cl. A61M 11/00

(52) U.S. Cl. 128/200.14

(57) ABSTRACT

The invention relates to an inhalation therapy device having an aerosol generator (1) comprising a membrane (2) that creates fluid droplets from fluid present on one side and gives off said droplets as an aerosol on the other side when the membrane is vibrated, and furthermore, a vibration creation arrangement (3) that is connected to the membrane such that the membrane is vibrated when the vibration creation arrangement is stimulated to vibrate in the activation phases of the aerosol generator by a supplied activation signal. An activation device (10) that is connected to the vibration creation arrangement is designed to supply an activation signal to the vibration creation arrangement (3) in the activation phases (Eₓ) of the aerosol generator (1), in particular an alternating voltage at several successive intervals (I₁ . . . Iₙ) spaced apart from one another.

The invention relates to an inhalation therapy device comprising a membrane aerosol generator for nebulising a therapeutically effective liquid.

Different aerosol generators are used for the nebulisation of liquids for inhalation purposes. Nozzle nebulisers or ultrasonic nebulisers are, for example, used, however, as compared to the membrane aerosol generators of the type in question here, these have a long switch-on phase (>1 s) before an aerosol is generated and released.

Known from WO 93/10910 A is a membrane aerosol generator comprising a membrane and an oscillation generating means. The oscillation generating means is a piezo oscillator, to which an actuation signal is supplied, by means of which the piezo oscillator is caused to oscillate. Since the piezo oscillator is connected to the membrane, the membrane is also caused to oscillate such that a liquid present on the one side of the membrane is conveyed through the openings in the membrane and is released on the other side of the membrane in the form of an aerosol. The actuation signal for the piezo oscillator is an a.c. voltage which is supplied to the piezo oscillator of the aerosol generator by an actuation circuit when an aerosol is supposed to be generated.

Membrane aerosol generators of this type are advantageously used in inhalation therapy nebulisers since the aerosol generator can be easily switched on and off by means of the actuation circuit. In this regard, the time periods in which an actuation signal is supplied to the aerosol generator of the inhalation therapy nebuliser are referred to as the switched-on phases of the aerosol generator. A distinction is thereby normally made between two operating modes.

In an inhalation therapy nebuliser which functions continuously, the switched-on phase of the aerosol generator regularly commences right at the beginning of a therapy session and is started by activation of aerosol generation by the patient (switching-on of the nebuliser). The switched-on phase of the aerosol generator ends when the patient interrupts aerosol generation (switching-off of the nebuliser) or when the liquid to be nebulised has been consumed.

In an inhalation therapy nebuliser which is controlled by a patient's breathing, the switched-on phase is regarded as that period which is determined by a control device detecting the respiration of the patient. The control device starts aerosol generation (switching-on of the aerosol generator) at a point determined by the patient's breathing and interrupts aerosol generation (switching-off of the aerosol generator) at another point determined by the patient's breathing. With this type of control, several successive switched-on phases that are adapted to the respiration, normally the inhalation phases, of the patient occur during a therapy session.

During the switched-on phase or phases, the aerosol generator generates an aerosol from liquid droplets that are presented to the user of the inhalation therapy device as an aerosol for inhalation.

Aerosol generators having a membrane and an oscillation generating means are particularly suitable for inhalation therapy purposes since the aerosol generated by these aerosol generators is of a particularly high and consistent quality. The reason for this is that a very high proportion of the generated liquid droplets are present in membrane aerosol generators in a respirable size of 0.1 to 10 μm (MMD) and preferably 0.5 to 5 μm (MMD). The total output rate (=TOR) is furthermore high, and thus therapy sessions that use an inhalation therapy nebuliser having a membrane aerosol generator are, as a rule, comparatively short. In many cases, this is advantageous and desirable since the patient only has to undergo a short therapy session. However, in other cases, a longer therapy session is desirable, but this should not be accompanied by an increased dosage. In further cases, the duration of the therapy sessions should stay the same but the dosage which is respectively administered should be different. In view of these fundamental requirements, it would be desirable if the amount of liquid released as an aerosol per unit of time could be variably reduced. However, this influencing of the release rate of the aerosol generator may not be accompanied by a deterioration in the quality of the aerosol.

Other considerations also argue in favour of a targeted reduction of the dose of the medicament aerosol (TOR) during inhalation therapy. Some of these considerations are summarised in the following:

Prevention of an irritation (overdosage) of all or regional (local) air passages, such as, for example:
irritation of the pharyngeal cavity and the glottis, which can cause coughing;
local overdosage of the lungs, for example: at branching sites (bifurcation) or in specific areas of the respiratory tract (trachea, bronchi, bronchioles and alveoli) or possible constrictions of the airways (obstructions), thus causing an undesirable increased side effect and/or damage to the lung tissue.

Reduction of the medicament output rate (TOR), for example in the neonatal treatment of premature babies having small respiratory volumes and high respiratory rates in order to enable a precise application in the small lungs and to achieve as efficient a deposition as possible of the active ingredient, such as, for example, an expensive “surfactant”.

Prevention of local hyperreactivity, irritations and lung tissue damage in connection with immunosuppressants during lung transplants. Possible active ingredients are glucocorticoids, cytostatic agents (Alkylating agents, anti-metabolites, intercalating agents), antibodies, modified immunophilins and other substances.

Provision of an adequate medicament dosage in tests carried out within the framework of authorisations for medicaments and substances for use in animals for general treatment at veterinary practices or for toxicological studies (TOX); specifically for the lungs of smaller animals such as mice, rats and dogs. Use in other animals, such as horses, is also possible.

Testing the lungs of a patient for hypersensitivity of the bronchial system (hyperreactive bronchial system) in non-specific or specific provocation tests for lung diagnosis. Histamine, methacholine or an allergen, for example, is thereby inhaled in increasing doses. The first doses of the substance are thereby often very small and should be applied safely and with a precise dosage.

Administration of new, very potent medicaments with a longer duration of inhalation therapy for safety reasons and for increased dosage accuracy.

Protection of persons who are indirectly involved in the inhalation therapy, such as relatives or medical personnel in hospitals and medical practices.

Against this background, the technical problem to be solved by the invention consists of creating an inhalation therapy nebuliser having a membrane aerosol generator, in which the amount of liquid released as an aerosol per unit of time can be influenced without affecting the quality of the aerosol.

This problem is solved by an inhalation therapy device having the features specified in patent claim 1. Advantageous embodiments can be found in the sub-claims.

According to the invention, the actuation signal, which causes the oscillation generating means of the aerosol generator to oscillate and which is generally and also preferably an a.c. voltage, is supplied during the switched-on phases of the aerosol generator in comparatively short intervals that follow one another with short gaps therebetween. Advantageous values for the control phases ON/OFF are in the range of 10 ms to 350 ms, preferably 10 to 150 ms, for the ON phase, and 10 ms to 500 ms, preferably 10 to 250 ms, for the OFF phase. This interval-type supply of the actuation signal/a.c. voltage and the selection of suitable relations for the ON/OFF phases during the switched-on phases allow the amount of liquid released as an aerosol per unit of time, and thus the total output rate (=TOR), to be influenced by amending the duty cycle of the intervals. If the intervals during which the actuation signal/a.c. signal is supplied are extended and the gaps between the intervals are shortened, the output rate is increased; if the intervals are shortened and the gaps between the intervals are lengthened, the output rate is reduced.

It is thereby quite surprising that the quality of the aerosol released by the membrane aerosol generator does not change or deteriorate even though the aerosol generator is switched on and off in very quick succession. It could not be expected that this operating mode of the aerosol generator, which is unusual from a conventional point of view, would not lead to a change in or deterioration of the droplet spectrum (MMD, GSD), i.e. the quality of the aerosol.

In consideration of this fact, the solution according to the invention may also be drawn upon to compensate for production-related variations, for example in respect of the number of holes in the membrane. A constant substance dose or TOR is thereby set for all membranes of a device specification (device configuration adapted to the medicament and patient group). If the device or aerosol generator is changed, this could be necessary in order to administer the new critical-dose substances with the same degree of accuracy. Changing the device may be necessary for various reasons and possible examples are device maintenance, repair, faults, cycle end and patient transfer from home use to use in hospital or a medical practice.

The solution according to the invention can be additionally drawn upon to compensate for modifications possibly brought about by aging processes occurring at the membrane. These modifications can manifest themselves in the form of a decreasing TOR over the life cycle of a membrane. With the help of the invention, a constant substance dosage can be set over the service life of the aerosol generator in applications having known aging processes. In order to do so, a reduced TOR is set from the outset, and with the aid of the invention, this TOR reduction is balanced out over time or the number of uses such that a constant TOR is on the whole provided over the entire lifetime.

The invention will be explained in more detail in the following by means of the drawings in which:

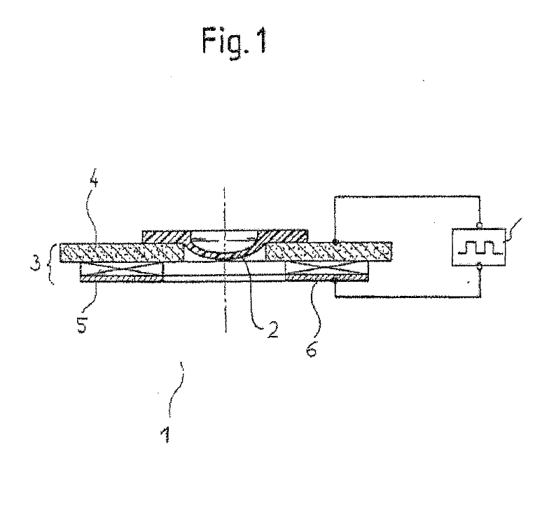

FIG. 1 shows the basic structure of a membrane aerosol generator;

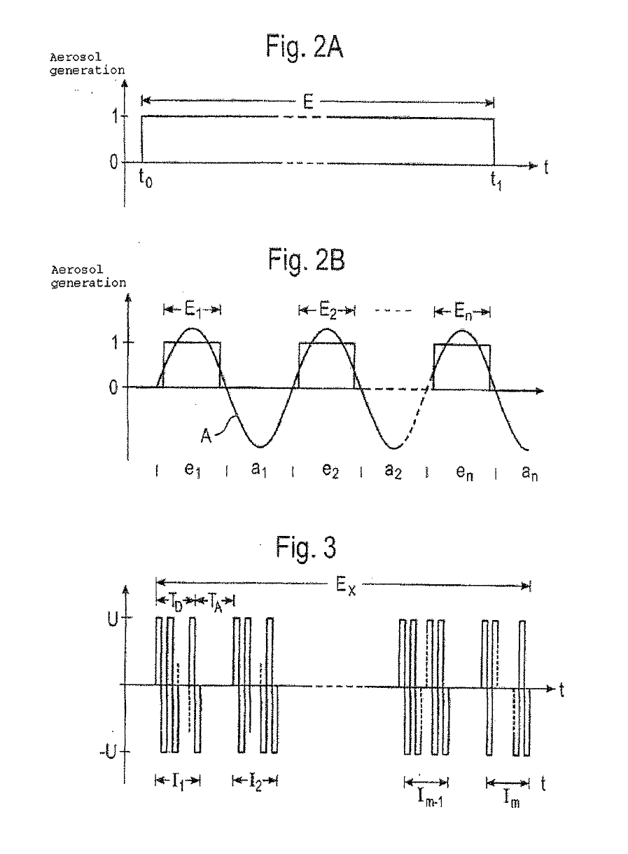

FIGS. 2A and 2B show the switched-on phases of the aerosol generator in continuous and respiration-controlled operation;

FIG. 3 shows an actuation signal as according to the invention for the aerosol generator;

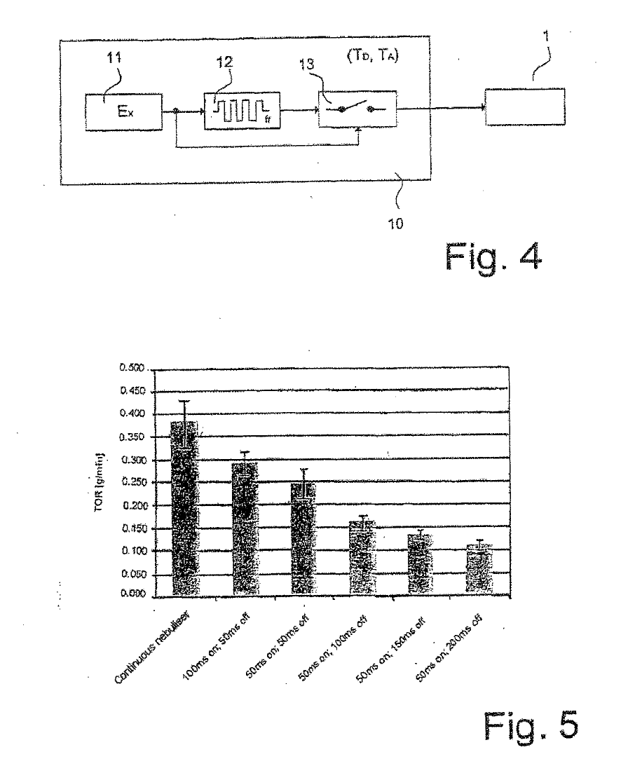

FIG. 4 shows a schematic representation of the relevant parts of an embodiment of an inhalation therapy nebuliser according to the invention; and

FIG. 5 shows a diagram portraying the measurement values achieved with an aerosol generator according to the invention.

Shown by way of example in FIG. 1 for a better understanding of the invention is the structure of the aerosol generator 1, which comprises a membrane 2 and an oscillation generator 3. The membrane shown in FIG. 1 is curved and has openings or holes in the region of the curve, through which a liquid passes when the membrane is caused to oscillate. For this purpose, the membrane is connected to the oscillation generator 3, which in the example shown in FIG. 1 is formed of a substrate 4, a piezo element 5 and an electrode 6. An a.c. voltage is supplied to the piezo element 5 via the conductive substrate 4 and the electrode 6, said voltage causing the oscillatable structure consisting of the piezo element and the substrate to oscillate and being provided by an actuation circuit 10. The oscillations are transferred to the membrane attached to the oscillatable structure, and thus the membrane is also caused to oscillate when an a.c. voltage is supplied to the piezo element.

The frequency of the supplied a.c. voltage is substantially determined by the resonance behaviour of the aerosol generator. In aerosol generators that are suitable for use in inhalation therapy nebulisers for the generation of an aerosol suitable for therapy, a.c. signals having frequencies of greater than 100 kHz are regularly used.

According to the invention, this a.c. voltage is not supplied continuously in the switched-on phases of the aerosol generator but is rather supplied to the oscillation generating means of the aerosol generator in several successive intervals that are spaced apart from one another. The actuation signal according to the invention is thus an a.c. voltage which, for the duration of the switched-on phase(s) of the aerosol generator, is supplied to the aerosol generator for actuation in several individual intervals in the magnitude of several milliseconds, which are separated from one another by several milliseconds. As an example, different intervals and gaps between these intervals were examined in a series of tests, and it was determined that with intervals of 10 to 150 ms at gaps of 10 to 250 ms, an accurate, predictable, linearly correlated reduction of the total output rate (=TOR) can be achieved whilst maintaining consistently good MMD values (aerosol quality). FIG. 5 shows by way of example the results of measurements carried out on 6 membrane aerosol generators. Continuous operation (continuous nebuliser), during which the actuation signal was supplied continuously, was thereby examined in comparison with the interval actuation according to the invention having the ON/OFF intervals shown in FIG. 5. The bars indicate the average value across the 6 aerosol generators, with the standard deviation for each average value also being shown in FIG. 5.

FIG. 2A shows the switched-on phase E of the aerosol generator in an inhalation therapy nebuliser, in which aerosol generation takes place continuously over the entire therapy session. Once the inhalation therapy nebuliser has been switched on, the actuation signal is supplied to the aerosol generator such that generation of the aerosol starts at time t₀ and ends at time t₁. This is often the point in time at which the liquid to be nebulised has been consumed. However, the patient can also end the therapy session of his own accord by switching off the inhalation therapy nebuliser. In the case of continuous aerosol generation, the switched-on phase E is independent of the patient's breathing.

By comparison, FIG. 2B shows the case of an inhalation therapy nebuliser with respiration-controlled aerosol generation. As is apparent from FIG. 2B, a control device (not shown) ensures that a plurality of switched-on phases E₁ . . . Eₙ are generated depending on the respiration of the patient, the course of which is indicated in FIG. 2B by curve A. FIG. 2B thereby shows the normal case wherein the switched-on phases E₁ . . . Eₙ occur in coordination with the inhalation phases e₁ . . . eₙ, whereas the aerosol generator is not switched on during the exhalation phases a₁ . . . aₙ.

FIG. 3 shows the actuation signal according to the invention by way of the example switched-on phase Eₓ, which is either the switched-on phase E according to FIG. 2A or one of switched-on phases E₁ . . . Eₙ according to FIG. 2B. It should thereby be noted that the representation in FIG. 3 is merely a schematic diagram provided so as to illustrate the nature of the actuation signal for the aerosol generator as according to the invention. The actuation signal according to the invention is in particular not limited to the rectangular a.c. signal shown in FIG. 3. It is apparent from FIG. 3 that during the switched-on phase Eₓ, an a.c. voltage is supplied to the aerosol generator in several successive intervals I₁ . . . Iₘ that are spaced apart from one another. According to the invention, the oscillation generating means and thus the membrane of the aerosol generator is ultimately only caused to oscillate in intervals I₁ . . . Iₘ, and thus aerosol is also only released in these intervals. The amount of aerosol released in the switched-on phase Eₓ, and thus the total output rate (=TOR) of the inhalation therapy nebuliser, can be influenced by the number of intervals during a switched-on phase Eₓ.

It is shown in FIG. 3 that the intervals I_{I} . . . Iₘ have the length T_{D} and that the gaps between these intervals have the length T_{A}. The rate of release can be modified by changing the values T_{D} and T_{A}. It is indicated in FIG. 3 that the values T_{D} and T_{A} are constant for a switched-on phase Eₓ. However, the values T_{D} and T_{A} can alternatively also be modified during a switched-on phase Eₓ.

FIG. 4 schematically shows the components of an inhalation therapy nebuliser, which are relevant here, according to an embodiment of the invention.

The aerosol generator is provided with reference number 1, the structure of which is shown in detail in FIG. 1. Reference is made to FIG. 1 in this respect.

An actuation signal, in this case an a.c. voltage, is supplied to the aerosol generator 1 by a control device 10, said actuation signal causing the oscillation means and thus the membrane of the aerosol generator 1 to oscillate such that an aerosol is generated. According to the invention, the control device 10 supplies the a.c. voltage during the switched-on phases of the aerosol generator in several successive intervals that are spaced apart from one another. For this purpose, the control device in the embodiment shown in FIG. 4 comprises a switched-on phase control means 11, which determines the switched-on phases Eₓ for continuous operation (FIG. 2A) or respiration-controlled operation (FIG. 2B). During these switched-on phases, an a.c. voltage generator 12 generates an actuation signal, for example a rectangular or sinusoidal (or other suitable) a.c. voltage, the frequency Fᵣ of which is adapted to the aerosol generator 1. The output signal of the a.c. voltage generator 12 is supplied to an interval switching means 13, which determines during a switched-on phase Eₓ the intervals and the gap therebetween, in which the a.c. voltage signal of the a.c. voltage generator 12 is supplied to the aerosol generator 1. For this purpose, the interval switching means 13 comprises default values for the length of the intervals T_{D} and the gap between the intervals T_{A}. During the switched-on phase Eₓ, the interval switching means 13 periodically connects the a.c. voltage signal of the a.c. voltage generator 12 through to the aerosol generator 1 over a time period T_{D} such that the aerosol generator outputs an aerosol in these intervals. The transmission of the a.c. voltage signal from the a.c. voltage generator 12 to the aerosol generator 1 is then subsequently interrupted for a time period T_{A}. During this time, the aerosol generator does not produce any aerosol. This transmission of the a.c. voltage signal from the a.c. voltage generator 12 to the aerosol generator 1 via the interval switching means 13, in each case for a period T_{D}, is periodically repeated throughout the entire switched-on phase Eₓ.

The amount of aerosol output by the aerosol generator 1 during a switched-on phase Eₓ can be defined by adjusting the interval length T_{D} and the interval gaps T_{A}. It can thereby be provided that the default values for the interval switching means 13 are infinitely selectable/adjustable or can be retrieved from a memory means.

1. An inhalation therapy device comprising
an aerosol generator (1) with
a membrane (2) which generates liquid droplets from a liquid present on the one side and releases these as an aerosol on the other side when the membrane is caused to oscillate, and
an oscillation generating means (3) that is connected to the membrane in such a manner that the membrane is caused to oscillate when the oscillation generating means is stimulated to oscillate during the switched-on phases of the aerosol generator by a supplied actuation signal, and
a control device (10) which is connected to the oscillation generating means,
characterised in that
the control device (10) is designed to supply an actuation signal, in particular an a.c. voltage, to the oscillation generating means (3) during the switched-on phases (Eₓ) of the aerosol generator (1) in a plurality of successive intervals (I₁ . . . Iₙ) which are spaced apart from one another.

2. An inhalation therapy device according to claim 1, characterised in that the control device (10) comprises an a.c. voltage generator (12) and an interval switching means (13), to which the a.c. voltage generated by the a.c. voltage generator is supplied and which transmits the a.c. voltage to the aerosol generator 1 in the intervals (I₁ . . . Iₙ).

3. An inhalation therapy device according to claim 2, characterised in that the length (T_{D}) of the intervals and the gaps (T_{A}) between these intervals (I₁ . . . Iₙ) are supplied to the interval switching means (13), and the length (T_{D}) preferably lies in the range of 10 ms to 350 ms, preferably 10 to 150 ms, and the gaps (T_{A}) preferably lie in the range of 10 ms to 500 ms, preferably 10 to 250 ms.

4. An inhalation therapy device according to claim 3, characterised in that the length (T_{D}) and the gaps (T_{A}) are variable, in particular infinitely adjustable.

5. An inhalation therapy device according to one of claims 1 to 4, characterised in that the control device (10) comprises a switched-on phase control means (11) which determines the switched-on phases of the aerosol generator (1).

6. An inhalation therapy device according to claim 5, characterised in that the switched-on phase control means (11) determines a continuous switched-on phase (E).

7. An inhalation therapy device according to claim 5, characterised in that the switched-on phase control means (11) determines a plurality of switched-on phases (E₁ . . . Eₙ) in a respiration-controlled manner.

8. An inhalation therapy device according to one of claims 1 to 7, characterised in that the oscillation generating means comprises a substrate (4) and a piezo element (5).
